(19) United States
(12) Patent Application Publication
(10) Pub. No.: US 2020/0246550 A1
(43) Pub. Date: Aug. 6, 2020

(54) SYRINGE FOR ADMINISTERING A PHARMACEUTICAL SUBSTANCE

(71) Applicant: Levete Wallace, Houston, TX (US)

(72) Inventor: Levete Wallace, Houston, TX (US)

(21) Appl. No.: 16/773,167

(22) Filed: Jan. 27, 2020

Related U.S. Application Data
(60) Provisional application No. 62/799,422, filed on Jan. 31, 2019.

(51) Int. Cl. A61M 5/315; A61M 5/32; A61M 5/19

(52) U.S. Cl. CPC A61M 5/3157; A61M 5/31511; A61M 5/3286; A61M 2005/3128; A61M 5/19; A61M 5/31596; A61M 5/3202

(57) ABSTRACT

Disclosed herein is a syringe for administering a pharmaceutical substance. Accordingly, the syringe may include a syringe body, an actuator, a dispensing needle, and a removable cap. Further, the syringe body may include a syringe wall. Further, the syringe wall forms a receptacle. Further, the receptacle may include the pharmaceutical substance associated with a predetermined dose. Further, the actuator may include a sliding plunger. Further, the sliding plunger may be configured for displacement through the receptacle. Further, the displacement of the sliding plunger causes dispensing of the predetermined dose of the pharmaceutical substance. Further, the dispensing needle may be detachably couplable with the syringe body proximal to a dispensing end of the syringe body. Further, the dispensing needle may include a conduit. Further, the removable cap may be attachable with the syringe body proximal to the dispensing end.

### RELATED APPLICATION(S)

Under provisions of 35 U.S.C. § 119e, the Applicant(s) claim the benefit of U.S. provisional application No. 62/799,422, titled “PRE-LOADED INSULIN SYRINGES”, filed on Jan. 31, 2019 which is incorporated herein by reference.

### TECHNICAL FIELD

Generally, the present disclosure relates to the field of health care instruments and supplies. More specifically, the present disclosure relates to a syringe for administering a pharmaceutical substance.

### BACKGROUND

Existing techniques for administering a pharmaceutical substance are deficient with regard to several aspects. For instance, it is challenging to administer the pharmaceutical substance in a correct dose using current technologies. Furthermore, the use of insulin injections by many diabetic patients has become a routine. Unfortunately, many diabetic patients are unable to reliably administer the correct dose. Also, in emergency situations, the time expended to properly prepare an accurate dose in a syringe can be critical.

Therefore, there is a need for an improved syringe for administering a pharmaceutical substance that may overcome one or more of the above-mentioned problems and/or limitations.

### BRIEF SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a syringe for administering a pharmaceutical substance, in accordance with some embodiments. Accordingly, the syringe may include a syringe body, an actuator, a dispensing needle, and a removable cap. Further, the syringe body may include a syringe wall. Further, the syringe wall forms a receptacle. Further, the receptacle may include the pharmaceutical substance associated with a predetermined dose. Further, the syringe body may include a syringe opening leading into the receptacle. Further, the actuator may include a sliding plunger. Further, the sliding plunger may be configured for displacement through the receptacle. Further, the displacement of the sliding plunger causes dispensing of the predetermined dose of the pharmaceutical substance. Further, the dispensing needle may be detachably couplable with the syringe body proximal to a dispensing end of the syringe body. Further, the dispensing needle may include a conduit. Further, the conduit may include a first end and a second end. Further, the first end may be attached to the dispensing end of the syringe body. Further, the first end may be configured for receiving the pharmaceutical substance from the syringe body. Further, the second end may be configured for penetrating skin. Further, the penetrating of the skin facilitates administering the pharmaceutical substance. Further, the removable cap may be attachable with the syringe body proximal to the dispensing end. Further, the removable cap may be configured to removably enclose the dispensing needle.

Further disclosed herein is a syringe for administering pharmaceutical substance, in accordance with some embodiments. Accordingly, the syringe may include a syringe body, an actuator, a dispensing nozzle, and a removable cap. Further, the syringe body may include a syringe wall. Further, the syringe wall forms a receptacle. Further, the receptacle stores the pharmaceutical substance associated with a predetermined dose. Further, the syringe body may include a syringe opening leading into the receptacle. Further, the actuator may include a sliding plunger. Further, the sliding plunger may be configured for displacement through the receptacle. Further, the displacement of the sliding plunger causes dispensing of the predetermined dose of the pharmaceutical substance. Further, the dispensing nozzle may be detachably couplable with the syringe body proximal to a dispensing end of the syringe body. Further, the dispensing nozzle may include a conduit. Further, the conduit may include a first end and a second end. Further, the first end may be attached to the dispensing end of the syringe body. Further, the first end may be configured for receiving the pharmaceutical substance from the syringe body. Further, the second end facilitates administering the pharmaceutical substance. Further, the removable cap may be attachable with the syringe body proximal to the dispensing end. Further, the removable cap may be configured to removably enclose the dispensing nozzle.

Further disclosed herein is a syringe for administering pharmaceutical substance, in accordance with some embodiments. Accordingly, the syringe may include a syringe body, an actuator, a dispensing needle, and a removable cap. Further, the syringe body may include a syringe wall. Further, the syringe wall forms a receptacle. Further, the receptacle stores the pharmaceutical substance associated with a predetermined dose. Further, the syringe body may include a syringe opening leading into the receptacle. Further, the syringe body may include a clip. Further, the clip may be configured for fastening the syringe body to a surface associated with a user. Further, the actuator may include a sliding plunger. Further, the sliding plunger may be configured for displacement through the receptacle. Further, the displacement of the sliding plunger causes dispensing of the predetermined dose of the pharmaceutical substance. Further, the dispensing needle may be detachably couplable with the syringe body proximal to a dispensing end of the syringe body. Further, the dispensing needle may include a conduit. Further, the conduit may include a first end and a second end. Further, the first end may be attached to the dispensing end of the syringe body. Further, the first end may be configured for receiving the pharmaceutical substance from the syringe body. Further, the second end may be configured for penetrating skin. Further, the penetrating of the skin facilitates administering the pharmaceutical substance. Further, the removable cap may be attachable with the syringe body proximal to the dispensing end. Further, the removable cap may be configured to removably enclose the dispensing needle.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a front view of a syringe for administering a pharmaceutical substance, in accordance with some embodiments.

FIG. 3 is a front view of the syringe for administering a pharmaceutical substance, in accordance with some embodiments.

FIG. 4 is a front view of the syringe for administering a pharmaceutical substance, in accordance with some embodiments.

FIG. 5 is a front view of the syringe for administering a pharmaceutical substance, in accordance with some embodiments.

FIG. 6 is a front view of a syringe for administering a pharmaceutical substance, in accordance with some embodiments.

FIG. 7 is a front view of the syringe for administering a pharmaceutical substance, in accordance with some embodiments.

FIG. 8 is a front view of the syringe for administering a pharmaceutical substance, in accordance with some embodiments.

FIG. 9 is a front view of the syringe for administering a pharmaceutical substance, in accordance with some embodiments.

FIG. 10 is a front view of a syringe for administering a pharmaceutical substance, in accordance with some embodiments.

FIG. 11 is a front view of the syringe for administering a pharmaceutical substance, in accordance with some embodiments.

FIG. 12 is a front view of the syringe for administering a pharmaceutical substance, in accordance with some embodiments.

FIG. 13 is a front view of the syringe for administering a pharmaceutical substance, in accordance with some embodiments.

FIG. 14 is a front view of a subcutaneous syringe, in accordance with some embodiments.

FIG. 15 is a rear view of the subcutaneous syringe, in accordance with some embodiments.

FIG. 16 is a front view of the subcutaneous syringe with the cap removed, in accordance with some embodiments.

FIG. 17 is a rear view of the subcutaneous syringe with the cap removed, in accordance with some embodiments.

FIG. 18 is a top right-side perspective view of the subcutaneous syringe, in accordance with some embodiments.

FIG. 19 is a rear top right-side perspective view of the subcutaneous syringe, in accordance with some embodiments.

FIG. 20 is a side view of an intravenous syringe, in accordance with some embodiments.

FIG. 21 is a front right-side perspective view of the intravenous syringe, in accordance with some embodiments.

FIG. 22 is a side view of an intravenous syringe, in accordance with some embodiments.

FIG. 23 is a side perspective view of the intravenous syringe, in accordance with some embodiments.

FIG. 24 is a side view of an intravenous syringe, in accordance with some embodiments.

FIG. 25 is a perspective view of the intravenous syringe, in accordance with some embodiments.

FIG. 26 is a side view of an intravenous syringe, in accordance with some embodiments.

FIG. 27 is a perspective view of the intravenous syringe, in accordance with some embodiments.

FIG. 28 is a side view of an intravenous syringe, in accordance with some embodiments.

FIG. 29 is a perspective view of the intravenous syringe, in accordance with some embodiments.

FIG. 30 is a block diagram of a computing device for implementing the methods disclosed herein, in accordance with some embodiments.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of a syringe for administering a pharmaceutical substance, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice-based interface, gesture-based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third-party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role-based access control, and so on.

Further, one or more steps of the method disclosed herein may be initiated, maintained, controlled and/or terminated based on a control input received from one or more devices operated by one or more users such as, for example, but not limited to, an end-user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human-readable secret data (e.g. username, password, passphrase, PIN, secret question, secret answer, etc.) and/or possession of a machine-readable secret data (e.g. encryption key, decryption key, bar codes, etc.) and/or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human-readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor, etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes a syringe for administering a pharmaceutical substance. Further, the disclosed syringe may be directed to a pre-loaded insulin syringe. Syringes may be provided in a range of sizes in 5-milliliter increments, and in both subcutaneous and intravenous embodiments. The syringes provide a safe and reliable dosage amount for diabetes patients who self-administer insulin injections and are also intended for emergency use.

The intravenous syringe is pre-loaded with an appropriate dosage of pre-mixed insulin solution and is provided in four sizes: a 5-milliliter intravenous syringe, a 10-milliliter intravenous syringe, a 15-milliliter intravenous syringe, and a 20-milliliter intravenous syringe.

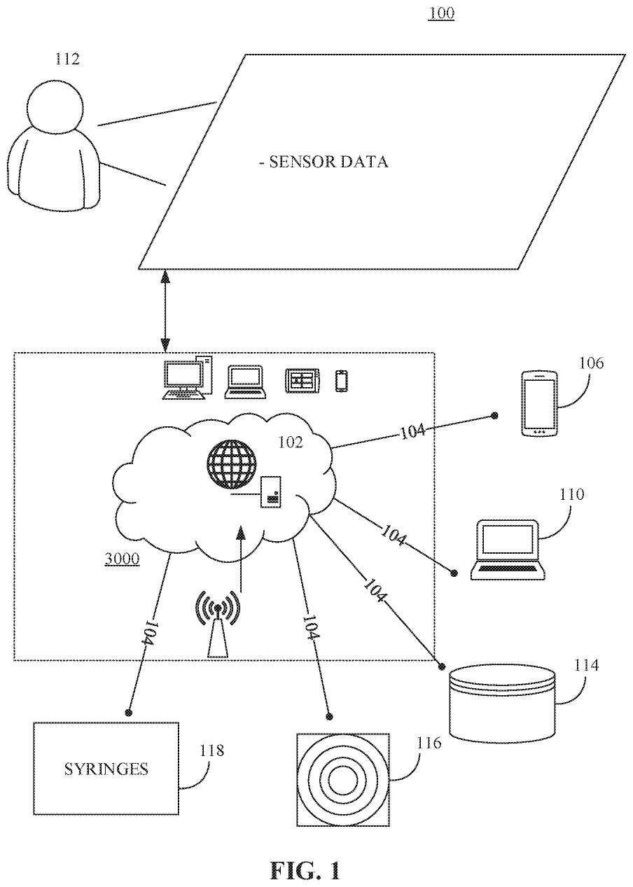

Referring now to figures, FIG. 1 is an illustration of an online platform 100 consistent with various embodiments of the present disclosure. By way of non-limiting example, the online platform 100 to facilitate the administration of a pharmaceutical substance may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, sensors 116, and syringes 118 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web-based software application or browser. The web-based software application may be embodied as, for example, but not be limited to, a website, a web application, a desktop application, and a mobile application compatible with a computing device 3000.

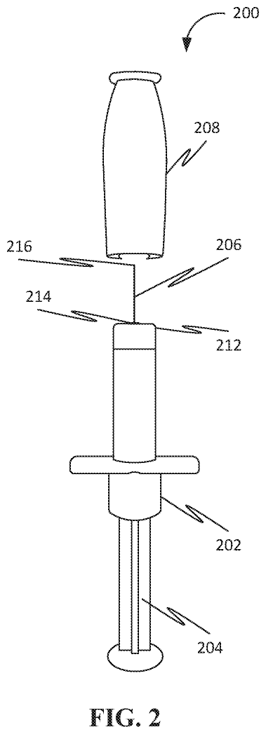

FIG. 2 is a front view of a syringe 200 for administering a pharmaceutical substance, in accordance with some embodiments. Accordingly, the syringe 200 may include a syringe body 202, an actuator 204, a dispensing needle 206, and a removable cap 208.

Further, the syringe body 202 may include a syringe wall (not shown). Further, the syringe wall forms a receptacle (not shown). Further, the receptacle may include the pharmaceutical substance associated with a predetermined dose. Further, the syringe body 202 may include a syringe opening (not shown) leading into the receptacle.

Further, the actuator 204 may include a sliding plunger (not shown). Further, the sliding plunger may be configured for displacement through the receptacle. Further, the displacement of the sliding plunger causes dispensing of the predetermined dose of the pharmaceutical substance.

Further, the dispensing needle 206 may be detachably couplable with the syringe body 202 proximal to a dispensing end 212 of the syringe body 202. Further, the dispensing needle 206 may include a conduit. Further, the conduit may include a first end 214 and a second end 216. Further, the first end 214 may be attached to the dispensing end 212 of the syringe body 202. Further, the first end 214 may be configured for receiving the pharmaceutical substance from the syringe body 202. Further, the second end 216 may be configured for penetrating the skin. Further, the penetrating of the skin facilitates administering the pharmaceutical substance.

Further, the removable cap 208 may be attachable with the syringe body 202 proximal to the dispensing end 212. Further, the removable cap 208 may be configured to removably enclose the dispensing needle 206.

Further, in some embodiments, the syringe 200 may be couplable with a surface using a fastening mechanism. Further, the surface may be associated with a user. Further, a first part of the fastening mechanism may be attachable with the syringe body 202. Further, a second part of the fastening mechanism may be attachable to the surface. Further, the first part and the second part are detachably couplable.

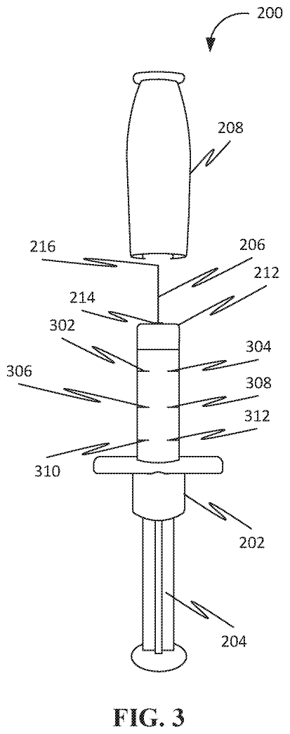

Further, in some embodiments, the syringe body 202 may include an inner surface proximal to the receptacle. Further, the inner surface may include a plurality of physical hindrances 302-312, as shown in FIG. 3. Further, each physical hindrance of the plurality of physical hindrances 302-312 may be configured to be arranged in a retracted state and an extended state. Further, the each physical hindrance allows the displacement of the sliding plunger in the retracted state. Further, the each physical hindrance restricts the displacement of the sliding plunger in the extended state. Further, the each physical hindrance may be configured for transitioning between the retracted state and the extended state based on action of a displacing force. Further, the displacing force facilitates the displacement of sliding plunger through the receptacle. Further, the receptacle may include a plurality of receptacle sections (not shown) corresponding to the plurality of physical hindrances 302-312. Further, the each receptacle section may include the pharmaceutical substance associated with a first predetermined dose.

Further, in some embodiments, the syringe body 202 may be associated with a size configured for storing quantity of the pharmaceutical substance from a group that may include 5 milliliters, 10 milliliters, 15 milliliters, and 20 milliliters.

Further, in some embodiments, the pharmaceutical substance may include a premixed insulin solution.

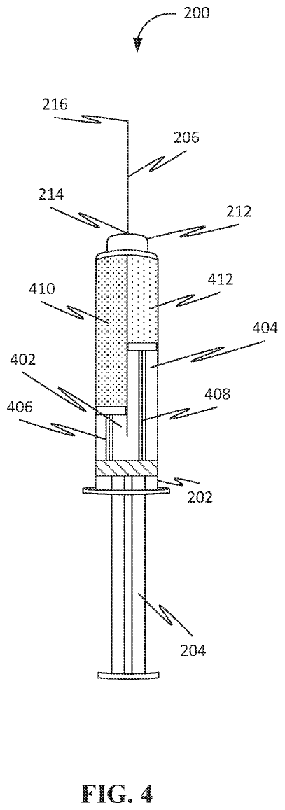

Further, in some embodiments, the syringe body 202 may include at least two compartments 402-404. Further, the at least two compartments 402-404 may include a first compartment 402 and a second compartment 404, as shown in FIG. 4. Further, the first compartment 402 may include a first plunger 406 and the second compartment 404 may include a second plunger 408. Further, the first compartment 402 may include a first receptacle 410. Further, the first receptacle 410 may include a first pharmaceutical substance (not shown) in a first predetermined dosage. Further, the second compartment 404 may include a second receptacle 412. Further, the second receptacle 412 may include a second pharmaceutical substance (not shown) in a second predetermined dosage. Further, the first receptacle 410 may include a first opening (not shown) and a first valve (not shown) disposed at the first opening. Further, the second receptacle 412 may include a second opening (not shown) and a second valve (not shown) disposed at the second opening. Further, the first opening may be in fluid communication with a primary opening (not shown) associated with the dispensing needle 206 through the first valve. Further, the second opening may be in fluid communication with the primary opening through the second valve. Further, the first valve may be characterized by a first threshold pressure. Further, the second valve may be characterized by a second threshold pressure. Further, the first plunger 406 and the second plunger 408 are operably attached to the sliding plunger associated with the syringe body 202. Further, the first plunger 406 may be characterized by a first length and the second plunger 408 may be characterized by a second length. Further, the first length, the second length, the first threshold pressure, and the second threshold pressure are configured to facilitate sequential dispensing of the first pharmaceutical substance and the second pharmaceutical substance based on the displacement of the sliding plunger.

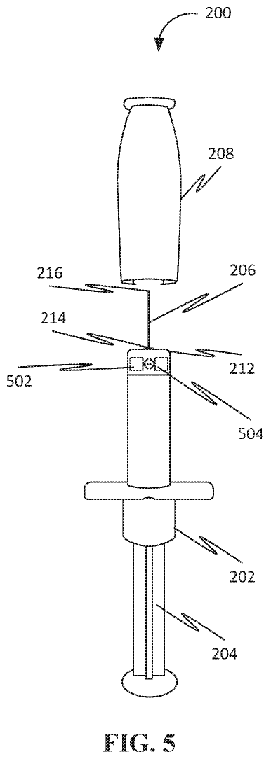

Further, in some embodiments, the syringe 200 may include a sensor 502, as shown in FIG. 5, disposed on the dispensing end 212 of the syringe body 202. Further, the syringe 200 further may include a processing device 504. Further, the sensor 502 may be communicatively coupled with the processing device 504. Further, the sensor 502 may be configured for detecting termination of dispensing of the predetermined dose of the pharmaceutical substance. Further, the sensor 502 may be configured for generating a sensor data. Further, the processing device 504 may be configured for analyzing the sensor data to generate an alert.

Further, in an embodiment, the syringe body 202 may include at least two compartments, wherein each compartment of the at least two compartments may include a compartment receptacle. Further, the compartment receptacle may include a medicinal substance associated with a dose corresponding to a user. Further, the syringe body may include a plunger corresponding to the each compartment. Further, each plunger is configured for displacement through the corresponding compartment receptacle. Further, the displacement of the each plunger causes dispensing of the medicinal substance from the each compartment receptacle. Further, the syringe body may include a valve. Further, the valve is configured for dispensing at least one of the medicinal substance from the each container receptacle.

FIG. 3 is a front view of the syringe 200 for administering a pharmaceutical substance, in accordance with some embodiments.

FIG. 4 is a front view of the syringe 200 for administering a pharmaceutical substance, in accordance with some embodiments.

FIG. 5 is a front view of the syringe 200 for administering a pharmaceutical substance, in accordance with some embodiments.

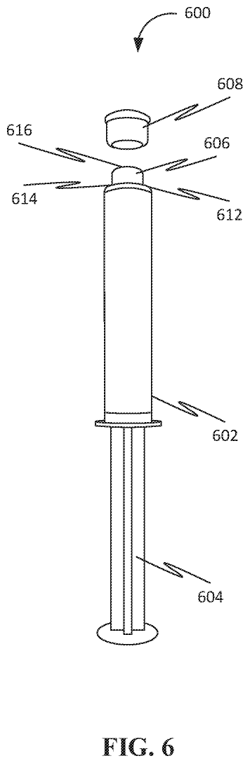

FIG. 6 is a front view of a syringe 600 for administering a pharmaceutical substance, in accordance with some embodiments. Accordingly, the syringe 600 may include a syringe body 602, an actuator 604, a dispensing nozzle 606, and a removable cap 608.

Further, the syringe body 602 may include a syringe wall (not shown). Further, the syringe wall forms a receptacle. Further, the receptacle stores the pharmaceutical substance associated with a predetermined dose. Further, the syringe body 602 may include a syringe opening (not shown) leading into the receptacle.

Further, the actuator 604 may include a sliding plunger (not shown). Further, the sliding plunger may be configured for displacement through the receptacle. Further, the displacement of the sliding plunger causes dispensing of the predetermined dose of the pharmaceutical substance.

Further, the dispensing nozzle 606 may be detachably couplable with the syringe body 602 proximal to a dispensing end 612 of the syringe body 602. Further, the dispensing nozzle 606 may include a conduit. Further, the conduit may include a first end 614 and a second end 616. Further, the first end 614 may be attached to the dispensing end 612 of the syringe body 602. Further, the first end 614 may be configured for receiving the pharmaceutical substance from the syringe body 602. Further, the second end 616 facilitates administering the pharmaceutical substance.

Further, the removable cap 608 may be attachable with the syringe body 602 proximal to the dispensing end 612. Further, the removable cap 608 may be configured to removably enclose the dispensing nozzle 606.

Further, in some embodiments, the syringe 600 may be couplable with a surface using a fastening mechanism. Further, the surface may be associated with a user. Further, a first part (not shown) of the fastening mechanism may be attachable with the syringe body 602. Further, a second part (not shown) of the fastening mechanism may be attachable to the surface. Further, the first part and the second part are detachably couplable.

Further, in some embodiments, the syringe body 602 may include an inner surface proximal to the receptacle. Further, the inner surface may include a plurality of physical hindrances 702-712. Further, each physical hindrance of the plurality of physical hindrances 702-712 may be configured to be arranged in a retracted state and an extended state. Further, the each physical hindrance allows the displacement of the sliding plunger in the retracted state. Further, the each physical hindrance restricts the displacement of the sliding plunger in the extended state. Further, the each physical hindrance may be configured for transitioning between the retracted state and the extended state based on action of a displacing force. Further, the displacing force facilitates the displacement of sliding plunger through the receptacle. Further, the receptacle may include a plurality of receptacle sections 714-718 corresponding to the plurality of physical hindrances 702-712. Further, the each receptacle section may include the pharmaceutical substance associated with a first predetermined dose.

Further, in some embodiments, the syringe body 602 may be associated with a size configured for storing a quantity of the pharmaceutical substance from a group that may include 5 milliliters, 10 milliliters, 15 milliliters, and 20 milliliters.

Further, in some embodiments, the pharmaceutical substance may include a premixed insulin solution.

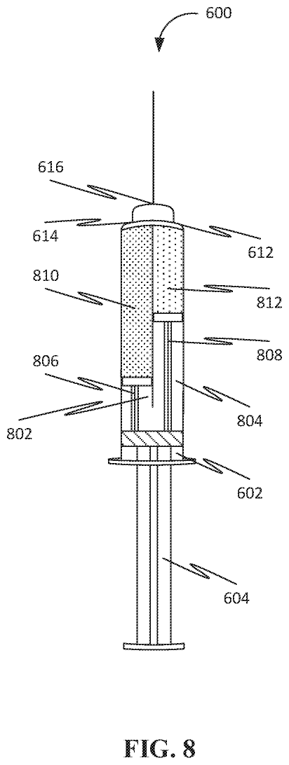

Further, in some embodiments, the syringe body 602 may include at least two compartments 802-804. Further, the at least two compartments 802-804 may include a first compartment 802 and a second compartment 804, as shown in FIG. 8. Further, the first compartment 802 may include a first plunger 806 and the second compartment 804 may include a second plunger 808. Further, the first compartment 802 may include a first receptacle 810. Further, the first receptacle 810 may include a first pharmaceutical substance (not shown) in a first predetermined dosage. Further, the second compartment 804 may include a second receptacle 812. Further, the second receptacle 812 may include a second pharmaceutical substance (not shown) in a second predetermined dosage. Further, the first receptacle 810 may include a first opening (not shown) and a first valve (not shown) disposed at the first opening. Further, the second receptacle 812 may include a second opening (not shown) and a second valve (not shown) disposed at the second opening. Further, the first opening may be in fluid communication with a primary opening associated with the dispensing needle through the first valve. Further, the second opening may be in fluid communication with the primary opening through the second valve. Further, the first valve may be characterized by a first threshold pressure. Further, the second valve may be characterized by a second threshold pressure. Further, the first plunger 806 and the second plunger 808 are operably attached to the sliding plunger associated with the syringe body 602. Further, the first plunger 806 may be characterized by a first length and the second plunger 808 may be characterized by a second length. Further, the first length, the second length, the first threshold pressure, and the second threshold pressure are configured to facilitate sequential dispensing of the first pharmaceutical substance and the second pharmaceutical substance based on the displacement of the sliding plunger.

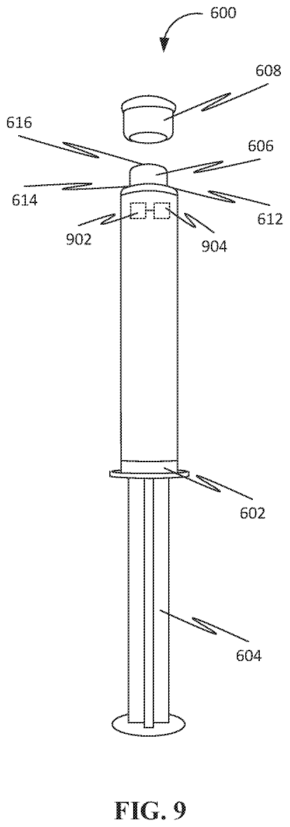

Further, in some embodiments, the syringe 600 may include a sensor 902, as shown in FIG. 9, disposed on the dispensing end 612 of the syringe body 602. Further, the syringe 600 further may include a processing device 904. Further, the sensor 902 may be communicatively coupled with the processing device 904. Further, the sensor 902 may be configured for detecting termination of dispensing of the predetermined dose of the pharmaceutical substance. Further, the sensor 902 may be configured for generating a sensor data. Further, the processing device 904 may be configured for analyzing the sensor data to generate an alert.

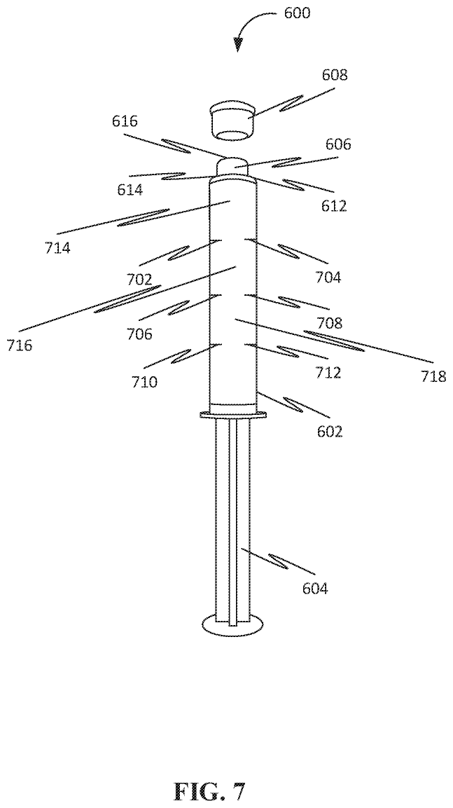

FIG. 7 is a front view of the syringe 600 for administering a pharmaceutical substance, in accordance with some embodiments.

FIG. 8 is a front view of the syringe 600 for administering a pharmaceutical substance, in accordance with some embodiments.

FIG. 9 is a front view of the syringe 600 for administering a pharmaceutical substance, in accordance with some embodiments.

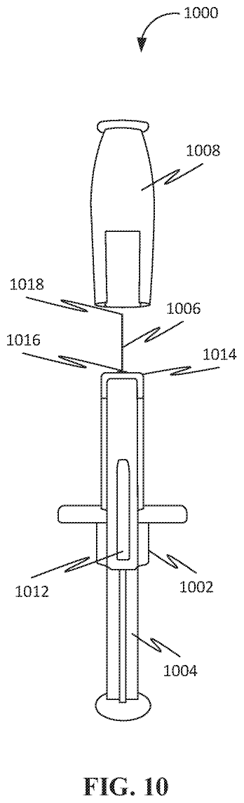

FIG. 10 is a front view of a syringe 1000 for administering a pharmaceutical substance, in accordance with some embodiments. Accordingly, the syringe 1000 may include a syringe body 1002, an actuator 1004, a dispensing needle 1006, and a removable cap 1008.

Further, the syringe body 1002 may include a syringe wall (not shown). Further, the syringe wall forms a receptacle (not shown). Further, the receptacle stores the pharmaceutical substance associated with a predetermined dose. Further, the syringe body 1002 may include a syringe opening (not shown) leading into the receptacle. Further, the syringe body 1002 may include a clip 1012. Further, the clip 1012 may be configured for fastening the syringe body 1002 to a surface associated with a user.

Further, the actuator 1004 may include a sliding plunger (not shown). Further, the sliding plunger may be configured for displacement through the receptacle. Further, the displacement of the sliding plunger causes dispensing of the predetermined dose of the pharmaceutical substance.

Further, the dispensing needle 1006 may be detachably couplable with the syringe body 1002 proximal to a dispensing end 1014 of the syringe body 1002. Further, the dispensing needle 1006 may include a conduit. Further, the conduit may include a first end 1016 and a second end 1018. Further, the first end 1016 may be attached to the dispensing end 1014 of the syringe body 1002. Further, the first end 1016 may be configured for receiving the pharmaceutical substance from the syringe body 1002. Further, the second end 1018 may be configured for penetrating the skin. Further, the penetrating of the skin facilitates administering the pharmaceutical substance.

Further, the removable cap 1008 may be attachable with the syringe body 1002 proximal to the dispensing end 1014. Further, the removable cap 1008 may be configured to removably enclose the dispensing needle 1006.

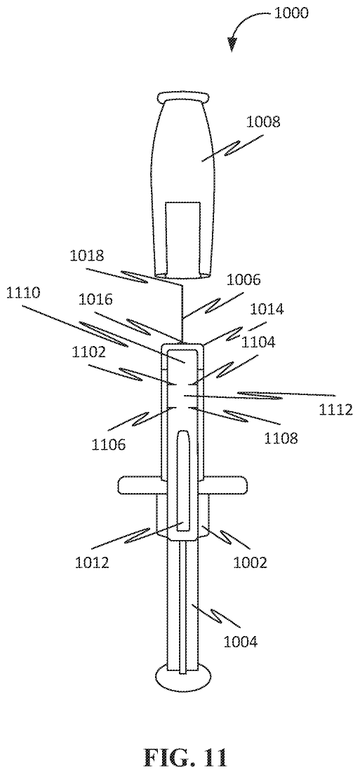

Further, in some embodiments, the syringe body 1002 may include an inner surface (not shown) proximal to the receptacle. Further, the inner surface may include a plurality of physical hindrances 1102-1108, as shown in FIG. 11. Further, each physical hindrance of the plurality of physical hindrances 1102-1108 may be configured to be arranged in a retracted state and an extended state. Further, the each physical hindrance allows the displacement of the sliding plunger in the retracted state. Further, the each physical hindrance restricts the displacement of the sliding plunger in the extended state. Further, the each physical hindrance may be configured for transitioning between the retracted state and the extended state based on action of a displacing force. Further, the displacing force facilitates the displacement of sliding plunger through the receptacle. Further, the receptacle may include a plurality of receptacle sections 1110-1112, as shown in FIG. 11, corresponding to the plurality of physical hindrances 1102-1108. Further, the each receptacle section may include the pharmaceutical substance associated with a first predetermined dose.

Further, in some embodiments, the syringe body 1002 may be associated with a size configured for storing quantity of the pharmaceutical substance from a group may include 5 milliliters, 10 milliliters, 15 milliliters, and 20 milliliters.

Further, in some embodiments, the pharmaceutical substance may include a premixed insulin solution.

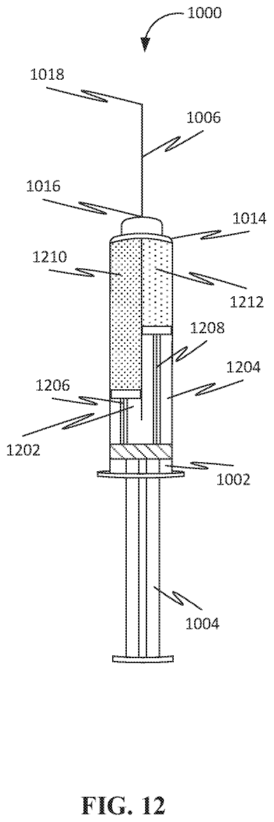

Further, in some embodiments, the syringe body 1002 may include at least two compartments 1202-1204. Further, the at least two compartments 1202-1204 may include a first compartment 1202 and a second compartment 1204, as shown in FIG. 12. Further, the first compartment 1202 may include a first plunger 1206 and the second compartment 1204 may include a second plunger 1208. Further, the first compartment 1202 may include a first receptacle 1210. Further, the first receptacle 1210 may include a first pharmaceutical substance in a first predetermined dosage. Further, the second compartment 1204 may include a second receptacle 1212. Further, the second receptacle 1212 may include a second pharmaceutical substance in a second predetermined dosage. Further, the first receptacle 1210 may include a first opening (not shown) and a first valve (not shown) disposed at the first opening. Further, the second receptacle 1212 may include a second opening (not shown) and a second valve (not shown) disposed at the second opening. Further, the first opening may be in fluid communication with a primary opening associated with the dispensing needle 1006 through the first valve. Further, the second opening may be in fluid communication with the primary opening through the second valve. Further, the first valve may be characterized by a first threshold pressure. Further, the second valve may be characterized by a second threshold pressure. Further, the first plunger 1206 and the second plunger 1208 are operably attached to the sliding plunger associated with the syringe body 1002. Further, the first plunger 1206 may be characterized by a first length and the second plunger 1208 may be characterized by a second length. Further, the first length, the second length, the first threshold pressure, and the second threshold pressure are configured to facilitate sequential dispensing of the first pharmaceutical substance and the second pharmaceutical substance based on the displacement of the sliding plunger.

Further, in some embodiments, the syringe 1000 may include a sensor 1302 disposed on the dispensing end 1014 of the syringe body 1002. Further, the syringe 1000 further may include a processing device 1304. Further, the sensor 1302 may be communicatively coupled with the processing device 1304. Further, the sensor 1302 may be configured for detecting termination of dispensing of the predetermined dose of the pharmaceutical substance. Further, the sensor 1302 may be configured for generating a sensor data. Further, the processing device 1304 may be configured for analyzing the sensor data to generate an alert.

FIG. 11 is a front view of the syringe 1000 for administering a pharmaceutical substance, in accordance with some embodiments.

FIG. 12 is a front view of the syringe 1000 for administering a pharmaceutical substance, in accordance with some embodiments.

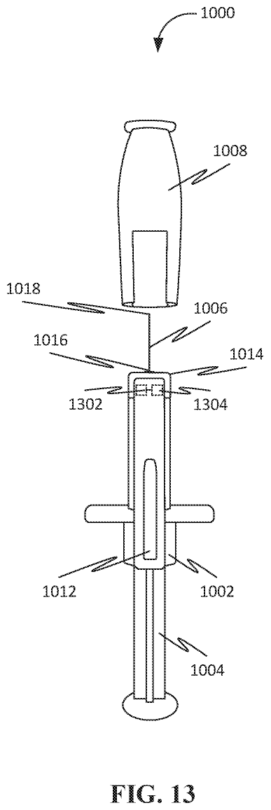

FIG. 13 is a front view of the syringe 1000 for administering a pharmaceutical substance, in accordance with some embodiments.

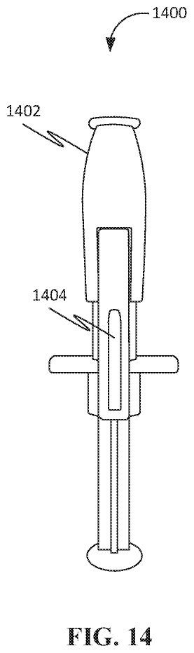

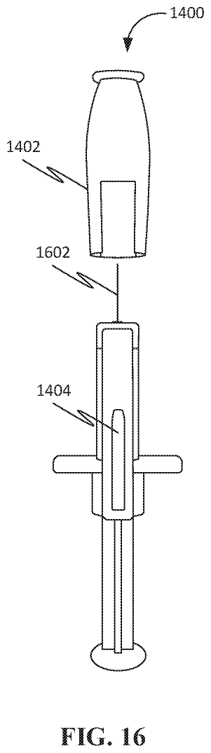

FIG. 14 is a front view of a subcutaneous syringe 1400, in accordance with some embodiments. The subcutaneous syringe 1400 may include a cap 1402, and a pocket clip 1404. The subcutaneous syringe 1400 may further include a needle 1602, as shown in FIG. 16. Further, the subcutaneous syringe 1400 includes the removable cap 1402, the pocket clip 1404, and the factory-installed subcutaneous needle 1602. The subcutaneous syringe 1400 may be pre-loaded with an appropriate dosage of pre-mixed insulin solution.

To use the subcutaneous syringe 1400, the user may remove the cap 1402, install a hypodermic needle if using the intravenous syringe 2000, and administer the injection. The subcutaneous syringe 1400 may be used in health institutions or outside health institutions. Currently, workers within health institutions have to withdraw insulin from a multi-use bottle/vial.

Specifically, the disclosed subcutaneous syringe 1400 with 5-milliliter, 10-milliliter, 15-milliliter, and 20-milliliter sizes may be used within health institutions or outside health institutions. However, the pre-diluted/bigger syringes which constitute 5-20 units of insulin diluted in normal saline to a maximum of 20 ml may be exclusively used within health institutions because it is prepared for intravenous use only. Insulin is measured in measurements of units which is a very minute quantity. Hence, for the bigger syringes 5 units would be added to 4.95 ml of normal saline to bring it to a total volume of 5 ml and same process is followed for 10 ml, 15 ml and 20 ml.

The cap 1402 is preferably manufactured from a flexible, durable material such as rubber or silicon. The needle 1602 is preferably manufactured from a rigid, durable material with substantial structural strength, such as stainless steel.

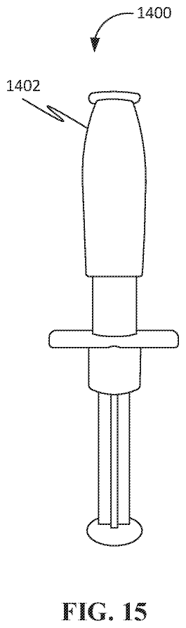

FIG. 15 is a rear view of the subcutaneous syringe 1400, in accordance with some embodiments.

FIG. 16 is a front view of the subcutaneous syringe 1400 with the cap 1402 removed, in accordance with some embodiments.

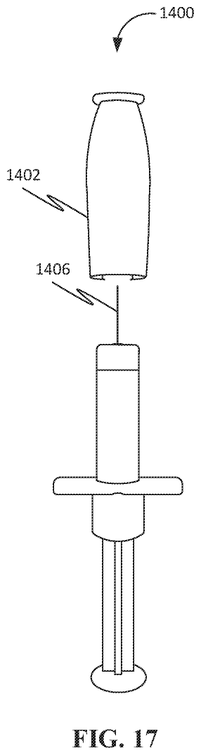

FIG. 17 is a rear view of the subcutaneous syringe 1400 with the cap 1402 removed, in accordance with some embodiments.

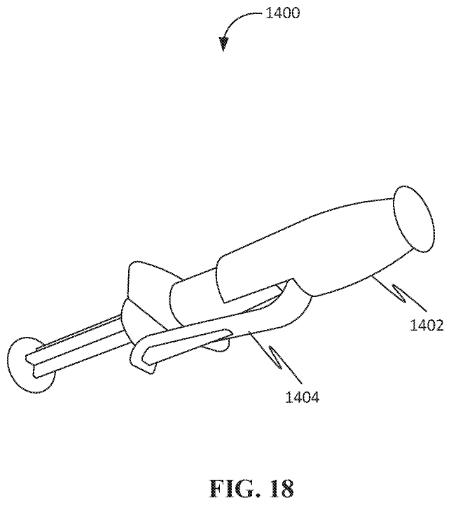

FIG. 18 is a top right-side perspective view of the subcutaneous syringe 1400, in accordance with some embodiments.

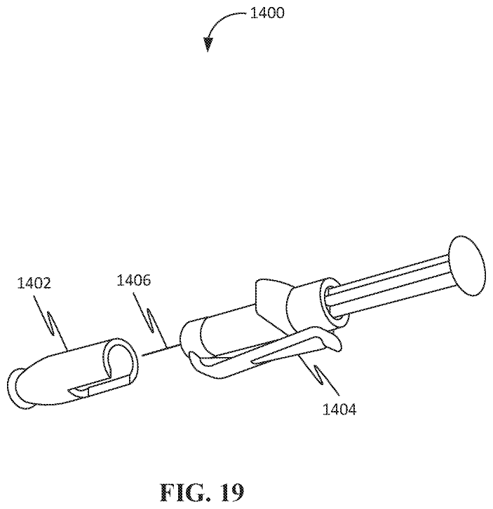

FIG. 19 is a rear top right-side perspective view of the subcutaneous syringe 1400, in accordance with some embodiments.

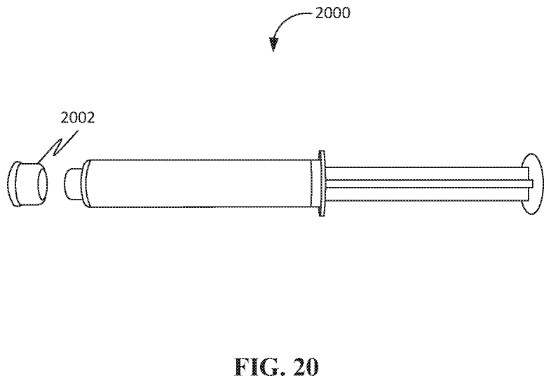

FIG. 20 is a side view of an intravenous syringe 2000, in accordance with some embodiments. The intravenous syringe 2000 may not include a factory-installed needle or a pocket clip. For example, the intravenous syringe 2000 may be associated with a 20-milliliter size.

To use the intravenous syringe 2000, the user may remove the cap 2002, install a hypodermic needle if using the intravenous syringe 2000, and administer the injection. Further, the cap 2002 is preferably manufactured from a flexible, durable material such as rubber or silicon.

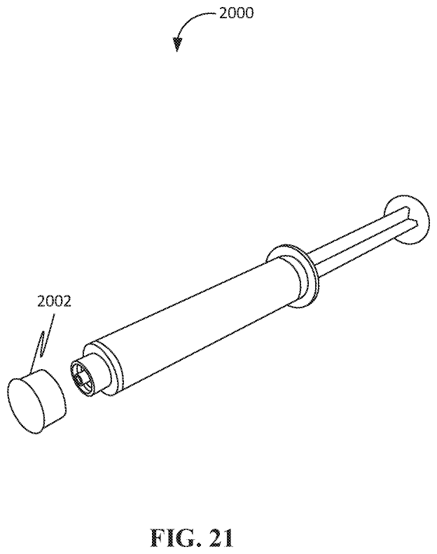

FIG. 21 is a front right-side perspective view of the intravenous syringe 2000, in accordance with some embodiments.

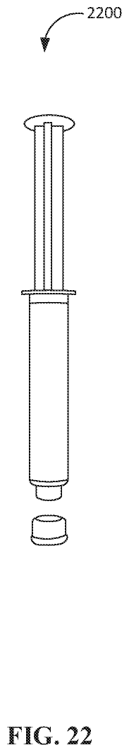

FIG. 22 is a side view of an intravenous syringe 2200, in accordance with some embodiments. Further, the intravenous syringe 2200 may be preferably manufactured from a rigid, disposable material such as plastic. For example, the intravenous syringes 2200 may have a 20-milliliter size.

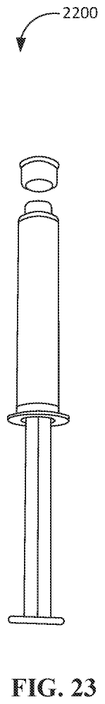

FIG. 23 is a side perspective view of the intravenous syringe 2200, in accordance with some embodiments.

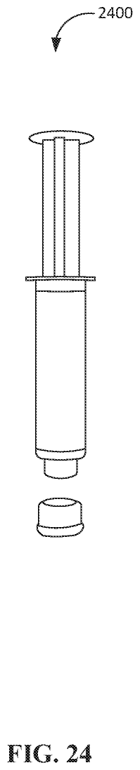

FIG. 24 is a side view of an intravenous syringe 2400, in accordance with some embodiments. Further, the intravenous syringe 2400 may be preferably manufactured from a rigid, disposable material such as plastic. For example, the intravenous syringes 2400 may have a 15-milliliter size.

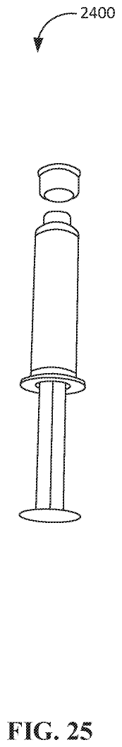

FIG. 25 is a perspective view of the intravenous syringe 2400, in accordance with some embodiments.

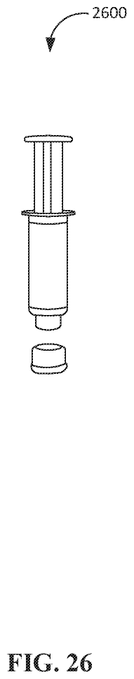

FIG. 26 is a side view of an intravenous syringe 2600, in accordance with some embodiments. Further, the intravenous syringe 2600 may be preferably manufactured from a rigid, disposable material such as plastic. For example, the intravenous syringes 2600 may have a 10-milliliter size.

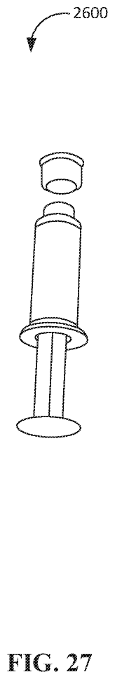

FIG. 27 is a perspective view of the intravenous syringe 2600, in accordance with some embodiments.

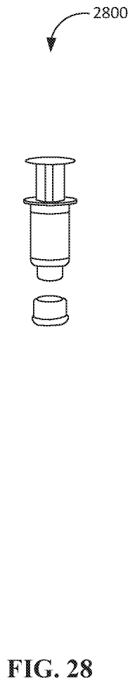

FIG. 28 is a side view of an intravenous syringe 2800, in accordance with some embodiments. Further, the intravenous syringe 2800 may be preferably manufactured from a rigid, disposable material such as plastic. For example, the intravenous syringes 2800 may have a 5-milliliter size.

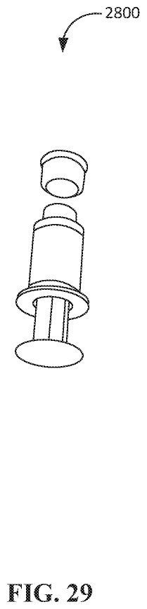

FIG. 29 is a perspective view of the intravenous syringe 2800, in accordance with some embodiments.

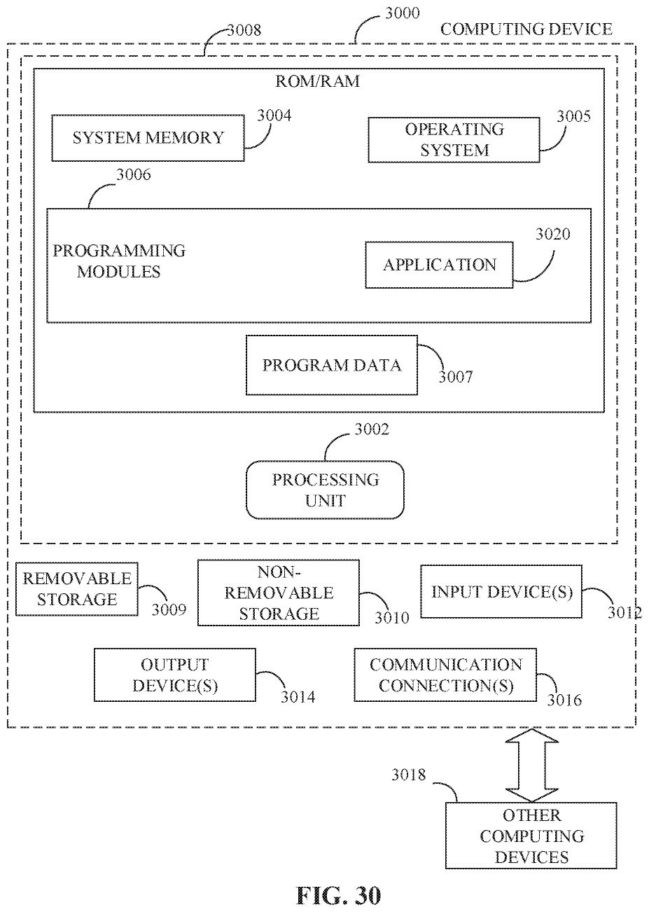

With reference to FIG. 30, a system consistent with an embodiment of the disclosure may include a computing device or cloud service, such as computing device 3000. In a basic configuration, computing device 3000 may include at least one processing unit 3002 and a system memory 3004. Depending on the configuration and type of computing device, system memory 3004 may comprise, but is not limited to, volatile (e.g. random-access memory (RAM)), non-volatile (e.g. read-only memory (ROM)), flash memory, or any combination. System memory 3004 may include operating system 3005, one or more programming modules 3006, and may include a program data 3007. Operating system 3005, for example, may be suitable for controlling computing device 3000's operation. Furthermore, embodiments of the disclosure may be practiced in conjunction with a graphics library, other operating systems, or any other application program and is not limited to any particular application or system. This basic configuration is illustrated in FIG. 30 by those components within a dashed line 3008.

Computing device 3000 may have additional features or functionality. For example, computing device 3000 may also include additional data storage devices (removable and/or non-removable) such as, for example, magnetic disks, optical disks, or tape. Such additional storage is illustrated in FIG. 30 by a removable storage 3009 and a non-removable storage 3010. Computer storage media may include volatile and non-volatile, removable and non-removable media implemented in any method or technology for storage of information, such as computer-readable instructions, data structures, program modules, or other data. System memory 3004, removable storage 3009, and non-removable storage 3010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 3000. Any such computer storage media may be part of device 3000. Computing device 3000 may also have input device(s) 3012 such as a keyboard, a mouse, a pen, a sound input device, a touch input device, a location sensor, a camera, a biometric sensor, etc. Output device(s) 3014 such as a display, speakers, a printer, etc. may also be included. The aforementioned devices are examples and others may be used.

Computing device 3000 may also contain a communication connection 3016 that may allow device 3000 to communicate with other computing devices 3018, such as over a network in a distributed computing environment, for example, an intranet or the Internet. Communication connection 3016 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein may include both storage media and communication media.

As stated above, a number of program modules and data files may be stored in system memory 3004, including operating system 3005. While executing on processing unit 3002, programming modules 3006 (e.g., application 3020 such as a media player) may perform processes including, for example, one or more stages of methods, algorithms, systems, applications, servers, databases as described above. The aforementioned process is an example, and processing unit 3002 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may, in fact, be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid-state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

The following is claimed:

1. A syringe for administering a pharmaceutical substance, wherein the syringe comprises:
a syringe body comprising a syringe wall, wherein the syringe wall forms a receptacle, wherein the receptacle comprises the pharmaceutical substance associated with a predetermined dose, wherein the syringe body comprises a syringe opening leading into the receptacle;
an actuator comprising a sliding plunger, wherein the sliding plunger is configured for displacement through the receptacle, wherein the displacement of the sliding plunger causes dispensing of the predetermined dose of the pharmaceutical substance;
a dispensing needle detachably couplable with the syringe body proximal to a dispensing end of the syringe body, wherein the dispensing needle comprises a conduit, wherein the conduit comprises a first end and a second end, wherein the first end is attached to the dispensing end of the syringe body, wherein the first end is configured for receiving the pharmaceutical substance from the syringe body, wherein the second end is configured for penetrating skin, wherein the penetrating of the skin facilitates administering the pharmaceutical substance; and
a removable cap attachable with the syringe body proximal to the dispensing end, wherein the removable cap is configured to removably enclose the dispensing needle.

2. The syringe of claim 1, wherein the syringe is couplable with a surface using a fastening mechanism, wherein the surface is associated with a user, wherein a first part of the fastening mechanism is attachable with the syringe body, wherein a second part of the fastening mechanism is attachable to the surface, wherein the first part and the second part are detachably couplable.

3. The syringe of claim 1, wherein the syringe body comprises an inner surface proximal to the receptacle, wherein the inner surface comprises a plurality of physical hindrances, wherein each physical hindrance of the plurality of physical hindrances is configured to be arranged in a retracted state and an extended state, wherein the each physical hindrance allows the displacement of the sliding plunger in the retracted state, wherein the each physical hindrance restricts the displacement of the sliding plunger in the extended state, wherein the each physical hindrance is configured for transitioning between the retracted state and the extended state based on action of a displacing force, wherein the displacing force facilitates the displacement of sliding plunger through the receptacle, wherein the receptacle comprises a plurality of receptacle sections corresponding to the plurality of physical hindrances, wherein the each receptacle section comprises the pharmaceutical substance associated with a first predetermined dose.

4. The syringe of claim 1, wherein the syringe body is associated with a size configured for storing quantity of the pharmaceutical substance from a group comprising 5 milliliters, 10 milliliters, 15 milliliters, and 20 milliliters.

5. The syringe of claim 1, wherein the pharmaceutical substance comprises a premixed insulin solution.

6. The syringe of claim 1, wherein the syringe body comprises at least two compartments, wherein the at least two compartments comprises a first compartment and a second compartment, wherein the first compartment comprises a first plunger and the second compartment comprises a second plunger, wherein the first compartment comprises a first receptacle, wherein the first receptacle comprises a first pharmaceutical substance in a first predetermined dosage, wherein the second compartment comprises a second receptacle, wherein the second receptacle comprises a second pharmaceutical substance in a second predetermined dosage, wherein the first receptacle comprises a first opening and a first valve disposed at the first opening, wherein the second receptacle comprises a second opening and a second valve disposed at the second opening, wherein the first opening is in fluid communication with a primary opening associated with the dispensing needle through the first valve, wherein the second opening is in fluid communication with the primary opening through the second valve, wherein the first valve is characterized by a first threshold pressure, wherein the second valve is characterized by a second threshold pressure, wherein the first plunger and the second plunger are operably attached to the sliding plunger associated with the syringe body, wherein the first plunger is characterized by a first length and the second plunger is characterized by a second length, wherein the first length, the second length, the first threshold pressure and the second threshold pressure are configured to facilitate sequential dispensing of the first pharmaceutical substance and the second pharmaceutical substance based on displacement of the sliding plunger.

7. The syringe of claim 1, wherein the syringe comprises a sensor disposed on the dispensing end of the syringe body, wherein the syringe further comprises a processing device, wherein the sensor is communicatively coupled with the processing device, wherein the sensor is configured for detecting termination of dispensing of the predetermined dose of the pharmaceutical substance, wherein the sensor is configured for generating a sensor data, wherein the processing device is configured for analyzing the sensor data to generate an alert.

8. A syringe for administering pharmaceutical substance, wherein the syringe comprises:
a syringe body comprising a syringe wall, wherein the syringe wall forms a receptacle, wherein the receptacle stores the pharmaceutical substance associated with a predetermined dose, wherein the syringe body comprises a syringe opening leading into the receptacle;
an actuator comprising a sliding plunger, wherein the sliding plunger is configured for displacement through the receptacle, wherein the displacement of the sliding plunger causes dispensing of the predetermined dose of the pharmaceutical substance;
a dispensing nozzle detachably couplable with the syringe body proximal to a dispensing end of the syringe body, wherein the dispensing nozzle comprises a conduit, wherein the conduit comprises a first end and a second end, wherein the first end is attached to the dispensing end of the syringe body, wherein the first end is configured for receiving the pharmaceutical substance from the syringe body, wherein the second end facilitates administering the pharmaceutical substance; and
a removable cap attachable with the syringe body proximal to the dispensing end, wherein the removable cap is configured to removably enclose the dispensing nozzle.

9. The syringe of claim 8, wherein the syringe is couplable with a surface using a fastening mechanism, wherein the surface is associated with a user, wherein a first part of the fastening mechanism is attachable with the syringe body, wherein a second part of the fastening mechanism is attachable to the surface, wherein the first part and the second part are detachably couplable.

10. The syringe of claim 8, wherein the syringe body comprises an inner surface proximal to the receptacle, wherein the inner surface comprises a plurality of physical hindrances, wherein each physical hindrance of the plurality of physical hindrances is configured to be arranged in a retracted state and an extended state, wherein the each physical hindrance allows the displacement of the sliding plunger in the retracted state, wherein the each physical hindrance restricts the displacement of the sliding plunger in the extended state, wherein the each physical hindrance is configured for transitioning between the retracted state and the extended state based on action of a displacing force, wherein the displacing force facilitates the displacement of sliding plunger through the receptacle, wherein the receptacle comprises a plurality of receptacle sections corresponding to the plurality of physical hindrances, wherein the each receptacle section comprises the pharmaceutical substance associated with a first predetermined dose.

11. The syringe of claim 8, wherein the syringe body is associated with a size configured for storing quantity of the pharmaceutical substance from a group comprising 5 milliliters, 10 milliliters, 15 milliliters, and 20 milliliters.

12. The syringe of claim 8, wherein the pharmaceutical substance comprises a premixed insulin solution.

13. The syringe of claim 8, wherein the syringe body comprises at least two compartments, wherein the at least two compartments comprises a first compartment and a second compartment, wherein the first compartment comprises a first plunger and the second compartment comprises a second plunger, wherein the first compartment comprises a first receptacle, wherein the first receptacle comprises a first pharmaceutical substance in a first predetermined dosage, wherein the second compartment comprises a second receptacle, wherein the second receptacle comprises a second pharmaceutical substance in a second predetermined dosage, wherein the first receptacle comprises a first opening and a first valve disposed at the first opening, wherein the second receptacle comprises a second opening and a second valve disposed at the second opening, wherein the first opening is in fluid communication with a primary opening associated with the dispensing needle through the first valve, wherein the second opening is in fluid communication with the primary opening through the second valve, wherein the first valve is characterized by a first threshold pressure, wherein the second valve is characterized by a second threshold pressure, wherein the first plunger and the second plunger are operably attached to the sliding plunger associated with the syringe body, wherein the first plunger is characterized by a first length and the second plunger is characterized by a second length, wherein the first length, the second length, the first threshold pressure and the second threshold pressure are configured to facilitate sequential dispensing of the first pharmaceutical substance and the second pharmaceutical substance based on displacement of the sliding plunger.

14. The syringe of claim 8, wherein the syringe comprises a sensor disposed on the dispensing end of the syringe body, wherein the syringe further comprises a processing device, wherein the sensor is communicatively coupled with the processing device, wherein the sensor is configured for detecting termination of dispensing of the predetermined dose of the pharmaceutical substance, wherein the sensor is configured for generating a sensor data, wherein the processing device is configured for analyzing the sensor data to generate an alert.

15. A syringe for administering pharmaceutical substance, wherein the syringe comprises:
a syringe body comprising a syringe wall, wherein the syringe wall forms a receptacle, wherein the receptacle stores the pharmaceutical substance associated with a predetermined dose, wherein the syringe body comprises a syringe opening leading into the receptacle, wherein the syringe body comprises a clip, wherein the clip is configured for fastening the syringe body to a surface associated with a user;
an actuator comprising a sliding plunger, wherein the sliding plunger is configured for displacement through the receptacle, wherein the displacement of the sliding plunger causes dispensing of the predetermined dose of the pharmaceutical substance;
a dispensing needle detachably couplable with the syringe body proximal to a dispensing end of the syringe body, wherein the dispensing needle comprises a conduit, wherein the conduit comprises a first end and a second end, wherein the first end is attached to the dispensing end of the syringe body, wherein the first end is configured for receiving the pharmaceutical substance from the syringe body, wherein the second end is configured for penetrating skin, wherein the penetrating of the skin facilitates administering the pharmaceutical substance; and
a removable cap attachable with the syringe body proximal to the dispensing end, wherein the removable cap is configured to removably enclose the dispensing needle.

16. The syringe of claim 15, wherein the syringe body comprises an inner surface proximal to the receptacle, wherein the inner surface comprises a plurality of physical hindrances, wherein each physical hindrance of the plurality of physical hindrances is configured to be arranged in a retracted state and an extended state, wherein the each physical hindrance allows the displacement of the sliding plunger in the retracted state, wherein the each physical hindrance restricts the displacement of the sliding plunger in the extended state, wherein the each physical hindrance is configured for transitioning between the retracted state and the extended state based on action of a displacing force, wherein the displacing force facilitates the displacement of sliding plunger through the receptacle, wherein the receptacle comprises a plurality of receptacle sections corresponding to the plurality of physical hindrances, wherein the each receptacle section comprises the pharmaceutical substance associated with a first predetermined dose.

17. The syringe of claim 15, wherein the syringe body is associated with a size configured for storing quantity of the pharmaceutical substance from a group comprising 5 milliliters, 10 milliliters, 15 milliliters, and 20 milliliters.

18. The syringe of claim 15, wherein the pharmaceutical substance comprises a premixed insulin solution.

19. The syringe of claim 15, wherein the syringe body comprises at least two compartments, wherein the at least two compartments comprises a first compartment and a second compartment, wherein the first compartment comprises a first plunger and the second compartment comprises a second plunger, wherein the first compartment comprises a first receptacle, wherein the first receptacle comprises a first pharmaceutical substance in a first predetermined dosage, wherein the second compartment comprises a second receptacle, wherein the second receptacle comprises a second pharmaceutical substance in a second predetermined dosage, wherein the first receptacle comprises a first opening and a first valve disposed at the first opening, wherein the second receptacle comprises a second opening and a second valve disposed at the second opening, wherein the first opening is in fluid communication with a primary opening associated with the dispensing needle through the first valve, wherein the second opening is in fluid communication with the primary opening through the second valve, wherein the first valve is characterized by a first threshold pressure, wherein the second valve is characterized by a second threshold pressure, wherein the first plunger and the second plunger are operably attached to the sliding plunger associated with the syringe body, wherein the first plunger is characterized by a first length and the second plunger is characterized by a second length, wherein the first length, the second length, the first threshold pressure and the second threshold pressure are configured to facilitate sequential dispensing of the first pharmaceutical substance and the second pharmaceutical substance based on displacement of the sliding plunger.

20. The syringe of claim 15, wherein the syringe comprises a sensor disposed on the dispensing end of the syringe body, wherein the syringe further comprises a processing device, wherein the sensor is communicatively coupled with the processing device, wherein the sensor is configured for detecting termination of dispensing of the predetermined dose of the pharmaceutical substance, wherein the sensor is configured for generating a sensor data, wherein the processing device is configured for analyzing the sensor data to generate an alert.
